(19) United States
(12) Patent Application Publication
(10) Pub. No.: US 2026/0224184 A1
(43) Pub. Date: Aug. 6, 2026

(54) IMAGE PROCESSING DEVICE, MEDICAL IMAGE CAPTURING SYSTEM, IMAGE PROCESSING METHOD, AND IMAGE PROCESSING PROGRAM

(71) Applicant: FUJIFILM Corporation, Tokyo (JP)

(72) Inventor: Rika TASHIRO, Tokyo (JP)

(73) Assignee: FUJIFILM Corporation, Tokyo (JP)

(21) Appl. No.: 19/572,829

(22) Filed: Mar. 19, 2026

Related U.S. Application Data
(63) Continuation of application No. PCT/JP2024/034812, filed on Sep. 27, 2024.

(30) Foreign Application Priority Data
Sep. 29, 2023 (JP) 2023-170679

(51) Int. Cl. A61B 6/00; A61B 6/04; A61B 6/50

(52) U.S. Cl. CPC A61B 6/5247; A61B 6/0414; A61B 6/502

(57) ABSTRACT

An image processing device including a processor configured to: acquire a radiographic image captured while a breast is in a compressed state by a compression member; acquire a plurality of ultrasound images captured while the breast is in a compressed state regarded as being the same as the compressed state of the radiographic image by the compression member; and superimpose a contour of the breast included in a radiographic image for superimposition corresponding to the radiographic image on an ultrasound image for display corresponding to the plurality of ultrasound images.

### CROSS REFERENCE TO RELATED APPLICATIONS

This application is a continuation of International Application No. PCT/JP2024/034812, filed on Sep. 27, 2024, which claims priority from Japanese Patent Application No. 2023-170679, filed on Sep. 29, 2023. The entire disclosure of each of the above applications is incorporated herein by reference.

### BACKGROUND

### Technical Field

The present disclosure relates to an image processing device, a medical image capturing system, an image processing method, and an image processing program.

### Related Art

In a case in which a radiographic image of a breast is observed, an ultrasound image of the breast is also referred to. For example, JP2005-125080A discloses a method of observing a region of interest in a radiographic image and a region of interest in an ultrasound image.

Meanwhile, in a case of continuously capturing the radiographic image and the ultrasound image while the breast is in a compressed state by a compression member, the radiographic image and the ultrasound image that are captured in the compressed state regarded as being the same may be interpreted. However, in the ultrasound image, a part of the breast may not be imaged, resulting in a missing image. For example, in a distal end part of the breast, the breast does not contact the compression member, and a region in which air is present between an ultrasound probe and the breast may be the missing image. In a case in which there is the missing image in the ultrasound image, it is difficult to compare the radiographic image with the ultrasound image.

### SUMMARY

The present disclosure provides an image processing device, a medical image capturing system, an image processing method, and an image processing program that can easily compare a radiographic image with an ultrasound cross-sectional image even in a case in which there is a missing image in the ultrasound image.

A first aspect of the present disclosure relates to an image processing device comprising: a processor configured to: acquire a radiographic image captured while a breast is in a compressed state by a compression member; acquire a plurality of ultrasound images captured while the breast is in a compressed state regarded as being the same as the compressed state of the radiographic image by the compression member; and superimpose a contour of the breast included in a radiographic image for superimposition corresponding to the radiographic image on an ultrasound image for display corresponding to the plurality of ultrasound images.

A second aspect of the present disclosure relates to the image processing device according to the first aspect, in which the processor is configured to receive designation of a depth of the breast, and the ultrasound image for display is a cross-sectional image that is combined from the plurality of ultrasound images, that corresponds to the received depth of the breast, and that is substantially parallel to an imaging table.

A third aspect of the present disclosure relates to the image processing device according to the first aspect, in which the ultrasound image for display is at least one of a first cross-sectional ultrasound image that is any one of the plurality of ultrasound images and that is a cross-sectional image in a direction intersecting an imaging table on which the breast is placed, a second cross-sectional ultrasound image that is combined from the plurality of ultrasound images and that is a cross-sectional image in a direction intersecting the imaging table and the first cross-sectional ultrasound image, or a third cross-sectional ultrasound image that is combined from the plurality of ultrasound images and that is a cross-sectional image substantially parallel to the imaging table.

A fourth aspect of the present disclosure relates to the image processing device according to the first aspect, in which the radiographic image is a projection image obtained by tomosynthesis imaging, and the radiographic image for superimposition is, among a plurality of radiation tomographic images obtained by reconstructing the projection image, the radiation tomographic image at a height corresponding to the ultrasound image for display.

A fifth aspect of the present disclosure relates to the image processing device according to the first aspect, in which the processor is configured to also display the radiographic image for superimposition including the contour of the breast.

A sixth aspect of the present disclosure relates to the image processing device according to the fifth aspect, in which the processor is configured to display the radiographic image for superimposition in a state of being superimposed with the contour of the breast included in the ultrasound image for display.

A seventh aspect of the present disclosure relates to the image processing device according to the first aspect, in which the processor is configured to further display a schematic diagram in which information representing a region corresponding to a difference between a region of the breast included in the ultrasound image for display and a region of the breast included in the radiographic image for superimposition is added.

An eighth aspect of the present disclosure relates to the image processing device according to the seventh aspect, in which the information includes a numerical value representing an interval between the contour of the breast included in the ultrasound image for display and the contour of the breast included in the radiographic image for superimposition.

A ninth aspect of the present disclosure relates to a medical image capturing system comprising: the image processing device according to the present disclosure; a radiographic image capturing apparatus; and an ultrasound image capturing apparatus.

A tenth aspect of the present disclosure relates to an image processing method executed by a processor provided in an image processing device, the image processing method comprising: acquiring a radiographic image captured while a breast is in a compressed state by a compression member; acquiring a plurality of ultrasound images captured while the breast is in a compressed state regarded as being the same as the compressed state of the radiographic image by the compression member; and superimposing a contour of the breast included in a radiographic image for superimposition corresponding to the radiographic image on an ultrasound image for display corresponding to the plurality of ultrasound images.

An eleventh aspect of the present disclosure relates to an image processing program causing a processor provided in an image processing device to execute a process comprising: acquiring a radiographic image captured while a breast is in a compressed state by a compression member; acquiring a plurality of ultrasound images captured while the breast is in a compressed state regarded as being the same as the compressed state of the radiographic image by the compression member; and superimposing a contour of the breast included in a radiographic image for superimposition corresponding to the radiographic image on an ultrasound image for display corresponding to the plurality of ultrasound images.

According to the present disclosure, it is possible to easily compare the radiographic image and the ultrasound cross-sectional image even in a case in which there is the missing image in the ultrasound image.

### BRIEF DESCRIPTION OF THE DRAWINGS

FIG. 1 is a configuration diagram schematically illustrating an example of an overall configuration of an image capturing system according to an embodiment.

FIG. 2 is a side view illustrating an example of an appearance of a mammography apparatus according to the embodiment.

FIG. 3 is a block diagram illustrating an example of a configuration of an ultrasound image capturing apparatus according to the embodiment.

FIG. 4 is a block diagram illustrating an example of a configuration of an image processing device according to the embodiment.

FIG. 5 is a functional block diagram illustrating an example of the configuration of the image processing device according to the embodiment.

FIG. 6 is a flowchart illustrating an example of a flow of capturing a radiographic image and the ultrasound image using an image capturing system according to the embodiment.

FIG. 7 is a flowchart illustrating an example of a flow of image processing in the image processing device according to the embodiment.

FIG. 8 is a diagram illustrating an example of a display state of an ultrasound cross-sectional image in a state in which a contour of a breast is superimposed and the radiographic image in the embodiment.

FIG. 9 is a diagram illustrating a missing image in the ultrasound image.

FIG. 10 is a diagram illustrating the missing image in the ultrasound image.

FIG. 11 is a side view illustrating an example of an appearance of an ultrasound probe.

FIG. 12 is a functional block diagram illustrating an example of a configuration of an image processing device of a modification example 1.

FIG. 13 is a diagram illustrating an example of a display state of an ultrasound cross-sectional image in a state in which a contour of a breast is superimposed and a radiation tomographic image in the modification example 1.

FIG. 14 is an example of a schematic diagram displayed in a modification example 2.

FIG. 15 is a diagram illustrating an example of a display state of an ultrasound cross-sectional image in a state in which a contour of a breast is superimposed and a radiographic image in a modification example 3.

### DETAILED DESCRIPTION

Hereinafter, embodiments of the present disclosure will be described in detail with reference to the accompanying drawings. The present disclosure is not limited to the present embodiment.

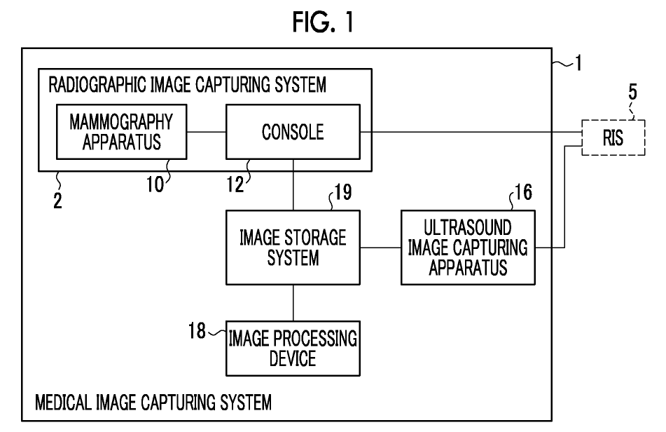

An example of an overall configuration of a medical image capturing system according to the present embodiment will be described first. FIG. 1 is a configuration diagram illustrating an example of an overall configuration of a medical image capturing system 1 according to the present embodiment.

As illustrated in FIG. 1, the medical image capturing system 1 according to the present embodiment comprises a radiographic image capturing system 2, an ultrasound image capturing apparatus 16, an image processing device 18, and an image storage system 19.

First, the configuration of the radiographic image capturing system 2 will be described. The radiographic image capturing system 2 includes a mammography apparatus 10 and a console 12.

The mammography apparatus 10 according to the present embodiment is an apparatus that uses a breast of an examinee as a subject and captures a radiographic image of the breast by irradiating the breast with radiation R (for example, X-rays). The mammography apparatus 10 may be an apparatus that images the breast of the examinee in a state (sitting state) in which the examinee is sitting on a chair (including a wheelchair) or the like, in addition to a state (standing state) in which the examinee is standing.

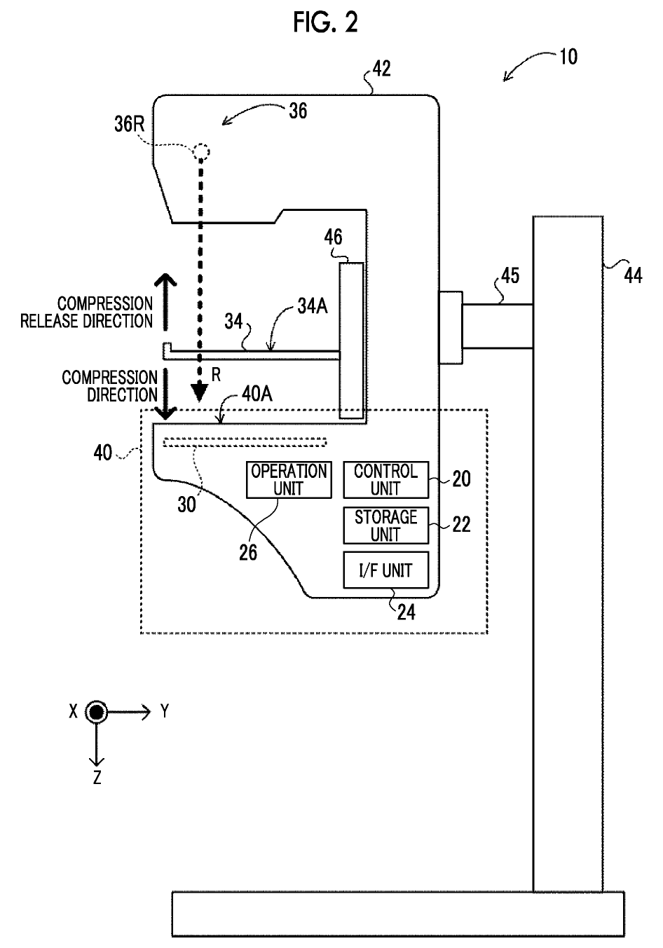

FIG. 2 is a side view illustrating an example of the appearance of the mammography apparatus 10 according to the present embodiment. FIG. 2 is a side view illustrating the mammography apparatus 10 as viewed from the right side of the examinee. As illustrated in FIG. 2, the mammography apparatus 10 includes a radiation source 36R, a radiation detector 30, an imaging table 40 disposed between the radiation source 36R and the radiation detector 30, and a compression member 34 that compresses the breast between the imaging table 40 and the compression member 34.

The imaging table 40 comprises a control unit 20, a storage unit 22, an interface (I/F) unit 24, an operation unit 26, and the radiation detector 30. The control unit 20 controls an overall operation of the mammography apparatus 10 in response to the control of the console 12. The control unit 20 comprises a central processing unit (CPU), a read-only memory (ROM), a random-access memory (RAM), and the like (not illustrated). The ROM stores, in advance, various programs including a program executed by the CPU for performing control related to radiographic image capturing. The RAM temporarily stores various data.

Image data of the radiographic image and various other types of information are stored in the storage unit 22. For example, the storage unit 22 is realized by storage media such as a hard disk drive (HDD), a solid-state drive (SSD), and a flash memory. Hereinafter, "image data of a radiographic image" is simply referred to as a "radiographic image". Further, similarly, "image data of an ultrasound image" is simply referred to as an "ultrasound image".

The I/F unit 24 communicates various types of information with the console 12 via wired communication or wireless communication. Specifically, the I/F unit 24 receives information on the control of the mammography apparatus 10 from the console 12. In addition, the I/F unit 24 transmits the radiographic image to the console 12.

The operation unit 26 is a part that is provided on the imaging table 40 or the like and that can be operated by a user with a hand, a foot, or the like, and is, for example, a switch, a button, a touch panel, or the like. For example, the operation unit 26 may receive voice input from the user.

The radiation detector 30 is disposed in the imaging table 40 to detect the radiation R transmitted through the breast that is the subject. In the mammography apparatus 10 according to the present embodiment, in a case in which the imaging is performed, the breast of the examinee is positioned on an imaging surface 40A of the imaging table 40 by the user such as a doctor or a radiology technician. For example, the imaging surface 40A or the like that is in contact with the breast of the examinee is made of carbon in terms of the transmittance or the intensity of the radiation R.

The radiation detector 30 detects the radiation R transmitted through the breast of the examinee and the imaging table 40, generates the radiographic image based on the detected radiation R, and outputs the generated radiographic image. The type of the radiation detector 30 according to the present embodiment is not particularly limited, and for example, the radiation detector 30 may be an indirect conversion type radiation detector that converts the radiation R into light and then converts the converted light into electric charges, or may be a direct conversion type radiation detector that directly converts the radiation R into electric charges.

The radiation source 36R is provided in a radiation irradiation unit 36. As illustrated in FIG. 2, the radiation irradiation unit 36 is provided on the arm part 42 together with the imaging table 40 and a compression unit 46. In addition, as illustrated in FIG. 2, the mammography apparatus 10 according to the present embodiment comprises the arm part 42, a base 44, and a shaft part 45. The arm part 42 is held by the base 44 to be movable in an up-down direction (Z-axis direction). The shaft part 45 connects the arm part 42 to the base 44. Further, the arm part 42 can be rotated relative to the base 44 using the shaft part 45 as a rotation axis.

Further, as illustrated in FIG. 2, the compression member 34 is attached to the compression unit 46. The compression unit 46 and the arm part 42 can be rotated relative to the base 44 separately using the shaft part 45 as a rotation axis. In the present embodiment, gears (not illustrated) are provided in each of the shaft part 45, the arm part 42, and the compression unit 46, and each gear is switched between an engaged state and a disengaged state to connect each of the arm part 42 and the compression unit 46 to the shaft part 45. One or both of the arm part 42 and the compression unit 46 connected to the shaft part 45 are rotated integrally with the shaft part 45.

The compression member 34 according to the present embodiment is a plate-shaped member, and is moved in the up-down direction (Z-axis direction) by a compression plate drive unit (not illustrated) provided in the compression unit 46 to compress the breast of the examinee between the compression member 34 and the imaging table 40. As illustrated in FIG. 2, regarding the movement direction of the compression member 34, a direction in which the breast is compressed, in other words, a direction in which the compression member 34 becomes closer to the imaging surface 40A is referred to as a "compression direction" and a direction in which the compression of the breast is released, in other words, a direction in which the compression member 34 becomes closer to the radiation irradiation unit 36 is referred to as a "compression release direction".

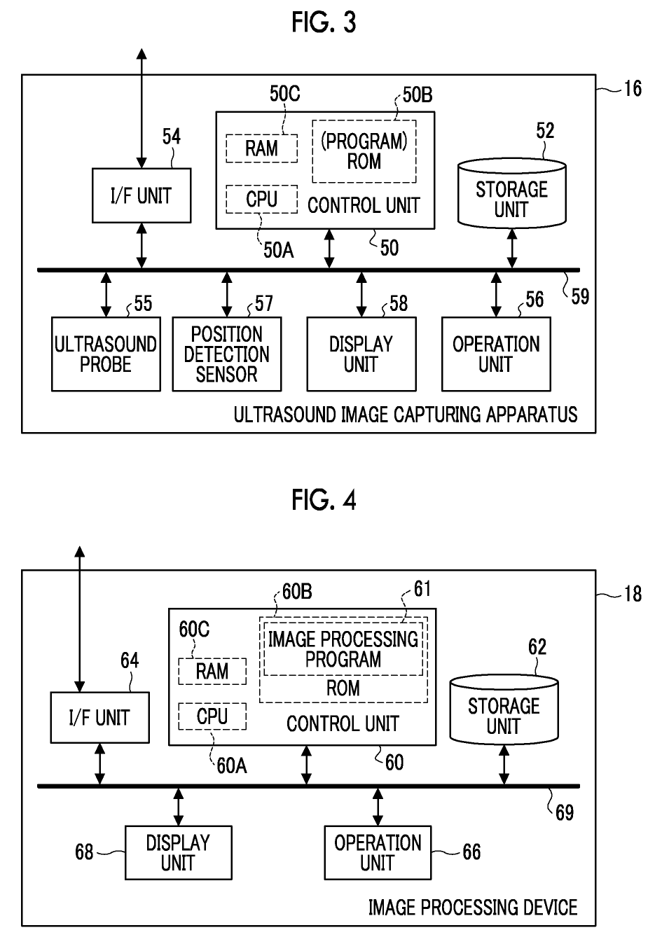

It is preferable that the compression member 34 is optically transparent in order to check positioning or the compressed state in a case of compressing the breast, and the compression member 34 is made of a material having excellent transmittance of the radiation R. Further, it is desirable that the compression member 34 is made of a material that facilitates the transmission of the ultrasound from an ultrasound probe 55 (see FIG. 3, details will be described later) of the ultrasound image capturing apparatus 16. Examples of the material of the compression member 34 include resins such as polymethylpentene, polycarbonate, acrylic, and polyethylene terephthalate. In particular, polymethylpentene is suitable as the material forming the compression member 34 since polymethylpentene has low rigidity, high elasticity, and high flexibility and has suitable values for acoustic impedance that affects the reflectance of the ultrasound and an attenuation coefficient that affects the attenuation of the ultrasound. The member constituting the compression member 34 is not limited to the member in the present embodiment. For example, the member constituting the compression member 34 may be a film-like member.

The compression member 34 is not limited to the compression member that compresses the entire breast, but may be a compression member that compresses a part of the breast. Stated another way, the compression member 34 may be smaller than the breast. As such a compression member 34, for example, the compression member 34 used for so-called spot imaging, in which the radiographic image is captured of only a region in which a lesion is present, is known.

Meanwhile, the console 12 according to the present embodiment has a function of controlling the mammography apparatus 10 using an imaging order and various types of information acquired from a radiology information system (RIS) 5 and the like through a wireless local area network (LAN), instructions input by the user using the operation unit 26, and the like. The console 12 according to the present embodiment is, as an example, a server computer.

Hereinafter, the configuration of the ultrasound image capturing apparatus 16 will be described. FIG. 3 is a block diagram illustrating an example of the configuration of the ultrasound image capturing apparatus 16. The ultrasound image capturing apparatus 16 is an apparatus that captures the ultrasound image with the breast of the examinee as the subject by the user, and is a so-called cart-type ultrasound image capturing apparatus. In addition, the ultrasound image capturing apparatus 16 may be a so-called compact type or a handheld type ultrasound image capturing apparatus other than the cart type ultrasound image capturing apparatus.

As illustrated in FIG. 3, the ultrasound image capturing apparatus 16 comprises a control unit 50, a storage unit 52, an I/F unit 54, an ultrasound probe 55, an operation unit 56, a position detection sensor 57, and a display unit 58. The control unit 50, the storage unit 52, the I/F unit 54, the ultrasound probe 55, the operation unit 56, the position detection sensor 57, and the display unit 58 are connected to each other through a bus 59, such as a system bus or a control bus, such that they can exchange various types of information with each other.

The control unit 50 according to the present embodiment controls the overall operation of the ultrasound image capturing apparatus 16. The control unit 50 comprises a CPU 50A, a ROM 50B, and a RAM 50C. The ROM 50B stores, in advance, various programs to be executed by the CPU 50A. The RAM 50C temporarily stores various data.

For example, the captured ultrasound image and various other types of information are stored in the storage unit 52. Specific examples of the storage unit 52 include an HDD and an SSD.

The ultrasound probe 55 is moved along an upper surface 34A (see FIG. 2, a surface opposite to the surface that comes into contact with the breast of the examinee) of the compression member 34 by the user, and scans the breast with the ultrasound to acquire the ultrasound image of the breast. Specifically, in a case in which the ultrasound imaging is performed, the ultrasound probe 55 is moved along the upper surface 34A of the compression member 34 by the user in a state in which an acoustic matching member (not illustrated) such as echo jelly is applied to the upper surface 34A of the compression member 34 or in a state in which the acoustic matching member is attached to the ultrasound probe 55.

The ultrasound probe 55 comprises a plurality of ultrasound transducers (see ultrasound transducers 55t in FIG. 11) arranged in a one-dimensional or two-dimensional shape. Each ultrasound transducer transmits the ultrasound based on an applied drive signal, receives an ultrasound echo, and outputs a reception signal.

Each of the plurality of ultrasound transducers is configured by, for example, a transducer in which electrodes are formed at both ends of a piezoelectric material (piezoelectric body), such as a piezoelectric ceramic represented by lead (Pb) zirconate titanate (PZT), or a polymer piezoelectric element represented by polyvinylidene difluoride (PVDF). In a case in which the pulsed or continuous wave drive signal is transmitted to apply the voltage to the electrodes of the transducer, the piezoelectric body is expanded and contracted. The pulsed or continuous wave ultrasound is generated from each transducer by these expansion and contraction, and these types of the ultrasound are combined to form an ultrasound beam. Further, each transducer receives the propagated ultrasound and then expanded and contracted to generate an electric signal. The electric signal is output as an ultrasound reception signal and is input to a body (not illustrated) of the ultrasound image capturing apparatus 16 via a cable (not illustrated).

The position detection sensor 57 is a sensor for detecting the position of the ultrasound probe 55. In the present embodiment, as the position detection sensor 57, a six-axis sensor that detects the movement direction, the orientation, and the rotation of the ultrasound probe 55 and that further calculates a movement distance, a movement speed, and the like is used. Specifically, the six-axis sensor is realized by a combination of an acceleration sensor that can detect three directions of the front-rear direction, the left-right direction, and the up-down direction and a geomagnetic sensor that can detect north, south, east, and west or a combination of the acceleration sensor and a gyro sensor that can detect the speed of rotation. The position detection sensor 57 is not limited to the six-axis sensor used in the present embodiment. For example, as the position detection sensor 57, a magnetic sensor as disclosed in JP2011-167331A may be used. Further, in the present embodiment, a method of using a sensor such as the position detection sensor 57 as a method of detecting the position of the ultrasound probe 55 is described, but the method of detecting the position of the ultrasound probe 55 is not limited to the method of using the sensor. For example, the position of the ultrasound probe 55 may be identified by attaching a marker that can detect a position in three axial directions in space to the ultrasound probe 55 and analyzing a captured image in which the marker is imaged. The position detection sensor 57 may be provided inside the ultrasound probe 55 or may be provided outside the ultrasound probe 55. Further, unlike the present embodiment, the position detection sensor 57 may be provided separately from the ultrasound image capturing apparatus 16. In any case, the position detection sensor 57 is not limited to such a disposition or configuration as long as the position detection sensor 57 can detect the position of the ultrasound probe 55.

The control unit 50 identifies a current position of the ultrasound probe 55. For example, the control unit 50 identifies the position of the ultrasound probe 55 with respect to the imaging surface 40A of the imaging table 40, the position of the ultrasound probe 55 with respect to the upper surface 34A of the compression member 34, or the position of the ultrasound probe 55 with respect to the body surface of the breast in the compressed state by the compression member 34.

In the medical image capturing system 1 according to the present embodiment, it is possible to associate any position in the ultrasound image captured by the ultrasound image capturing apparatus 16 with any pixel position in the radiographic image captured by the mammography apparatus 10 based on the position of the ultrasound probe 55. Therefore, the position of the ultrasound probe 55 detected by the position detection sensor 57 is added to the captured ultrasound image and then output.

The operation unit 56 is used by the user to input, for example, instructions or various types of information on the imaging of the ultrasound image. The operation unit 56 is not particularly limited, and examples of the operation unit 56 include various switches, a touch panel, a touch pen, and a mouse. The display unit 58 displays, for example, various types of information or the ultrasound image corresponding to the reception signal from the ultrasound probe 55. In addition, the operation unit 56 and the display unit 58 may be integrated to form a touch panel display.

The I/F unit 54 communicates various types of information with the RIS 5 and the image storage system 19 via wireless communication or wired communication. The ultrasound image captured by the ultrasound image capturing apparatus 16 is transmitted to the image storage system 19 via the I/F unit 54 through wireless communication or wired communication.

Hereinafter, the image storage system 19 will be described. The image storage system 19 is a system that stores the radiographic image captured by the radiographic image capturing system 2 and the ultrasound image captured by the ultrasound image capturing apparatus 16. The image storage system 19 is connected to each of the console 12 and the ultrasound image capturing apparatus 16 via wireless communication or wired communication. The image storage system 19 extracts an image corresponding to a request from, for example, the console 12, the ultrasound image capturing apparatus 16, and other image interpretation devices (not illustrated) from among the stored radiographic images and ultrasound images, and transmits the extracted image to the device which is the request source. A specific example of the image storage system 19 is picture archiving and communication systems (PACS).

Hereinafter, the image processing device 18 will be described. The image processing device 18 has a function of acquiring each of the radiographic image captured by the radiographic image capturing system 2 and the ultrasound image captured by the ultrasound image capturing apparatus 16 from the image storage system 19 and performing predetermined image processing.

FIG. 4 is a block diagram illustrating an example of the configuration of the image processing device 18. As illustrated in FIG. 4, the image processing device 18 comprises a control unit 60, a storage unit 62, an I/F unit 64, an operation unit 66, and a display unit 68. The control unit 60, the storage unit 62, the I/F unit 64, the operation unit 66, and the display unit 68 are connected to each other through a bus 69, such as a system bus or a control bus, such that they can exchange various types of information with each other.

The control unit 60 according to the present embodiment controls the overall operation of the image processing device 18. The control unit 60 comprises a CPU 60A, a ROM 60B, and a RAM 60C. The ROM 60B stores, in advance, various programs including an image processing program 61 (described later) which is executed by the CPU 60A. The RAM 60C temporarily stores various data.

The storage unit 62 stores, for example, the radiographic image, the ultrasound image, and various types of other information acquired from the image storage system 19. Specific examples of the storage unit 62 include an HDD and an SSD.

The operation unit 66 is used by the user to input, for example, instructions on image processing or various types of information. The operation unit 66 is not particularly limited, and examples of the operation unit 66 include various switches, a touch panel, a touch pen, and a mouse. The display unit 68 displays various types of information. In addition, the operation unit 66 and the display unit 68 may be integrated into a touch panel display.

The I/F unit 64 communicates the radiographic images, the ultrasound images, and various types of information with the image storage system 19 through wireless communication or wired communication.

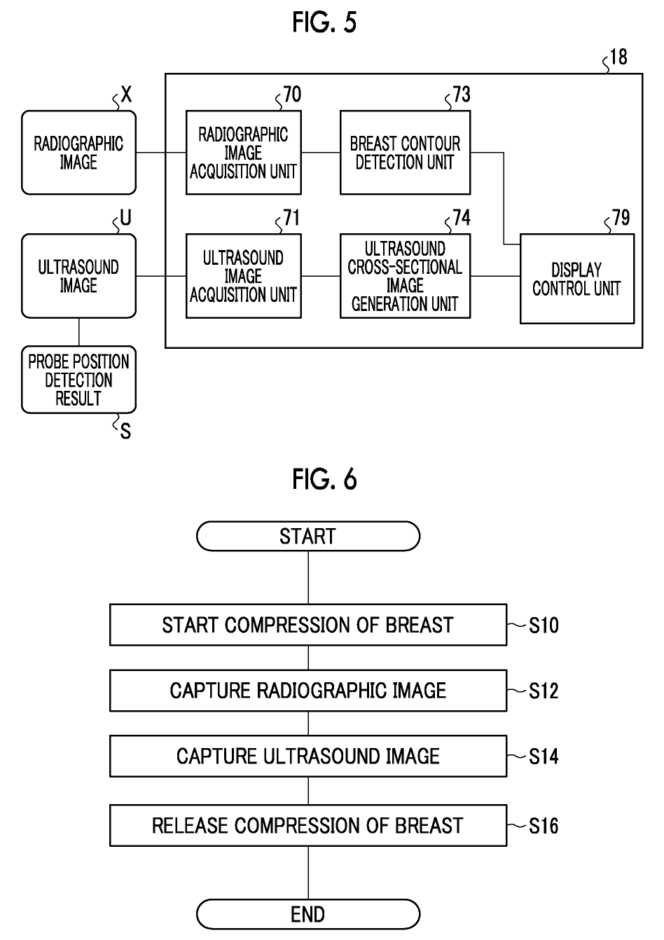

FIG. 5 is a functional block diagram illustrating an example of the function of the image processing device 18. The image processing device 18 comprises a radiographic image acquisition unit 70, an ultrasound image acquisition unit 71, a breast contour detection unit 73, an ultrasound cross-sectional image generation unit 74, and a display control unit 79. For example, in the image processing device 18 according to the present embodiment, the CPU 60A of the control unit 60 functions as the radiographic image acquisition unit 70, the ultrasound image acquisition unit 71, the breast contour detection unit 73, the ultrasound cross-sectional image generation unit 74, and the display control unit 79 by executing the image processing program 61.

The radiographic image acquisition unit 70 has a function of acquiring a radiographic image X. For example, in the present embodiment, a set of the radiographic image X and the ultrasound image U satisfying a display condition input by the user through the operation unit 66 and obtained by continuous imaging, which will be described in detail later, is acquired. Therefore, in a case in which the display condition input by the user is received, the radiographic image acquisition unit 70 acquires the radiographic image X in the set of the radiographic image X and the ultrasound image U corresponding to the received display condition from the image storage system 19 via the I/F unit 64. The display condition includes identification information that identifies the examinee and the breast, information indicating an imaging date and time, and identification information added for each set of imaging. The radiographic image acquisition unit 70 outputs the acquired radiographic image X to the breast contour detection unit 73.

The ultrasound image acquisition unit 71 has a function of acquiring the ultrasound image U. As described above, the ultrasound image acquisition unit 71 according to the present embodiment acquires, from the image storage system 19, the ultrasound image U corresponding to the display condition used in a case in which the radiographic image acquisition unit 70 acquires the radiographic image, via the I/F unit 64. In addition, in the ultrasound image capturing apparatus 16 according to the present embodiment, the ultrasound image U of the entire breast is captured by repeating imaging a plurality of times while performing scanning using the ultrasound probe 55. That is, a plurality of ultrasound images U continuously captured are obtained for the entire breast. Therefore, the ultrasound image acquisition unit 71 acquires the plurality of ultrasound images U. For simplifying the description, the plurality of ultrasound images U may be simply referred to as "ultrasound images U". The ultrasound image acquisition unit 71 outputs the acquired ultrasound image U to the ultrasound cross-sectional image generation unit 74.

That is, in the present embodiment, in the set of the radiographic image X and the ultrasound image U that are continuously captured, the radiographic image X is acquired by the radiographic image acquisition unit 70, and the ultrasound image U is acquired by the ultrasound image acquisition unit 71. A probe position detection result S is added to the ultrasound image U according to the present embodiment. The probe position detection result S is the detection result of the position detection sensor 57 of the ultrasound image capturing apparatus 16. Specifically, the detection result of detecting the position of the ultrasound probe 55 in a case of capturing the ultrasound image U with the position detection sensor 57 is added to each ultrasound image U as the probe position detection result S.

The breast contour detection unit 73 detects the contour of the breast included in the radiographic image X acquired by the radiographic image acquisition unit 70. A method in which the breast contour detection unit 73 detects the image of the breast included in the radiographic image X is not particularly limited. For example, the breast contour detection unit 73 may detect a position at which a density level difference in the radiographic image X is equal to or greater than a predetermined value, and detect a set of pixels having the density level difference that is equal to or greater than the predetermined value as the contour of the breast. In addition, for example, the breast contour detection unit 73 may perform segmentation on a region of the breast included in the radiographic image X, and detect a region corresponding to the image of the breast. The segmentation may be performed by detecting the contour of the breast by pattern matching, or may be performed by using a machine learning model such as a convolutional neural network (CNN).

The breast contour detection unit 73 outputs the detected contour of the breast from the radiographic image X and the radiographic image X to the display control unit 79. The radiographic image X according to the present embodiment is an example of a radiographic image for superimposition according to the present disclosure.

The ultrasound cross-sectional image generation unit 74 has a function of combining the plurality of ultrasound images U acquired by the ultrasound image acquisition unit 71 to generate an ultrasound cross-sectional image of a surface substantially parallel to the imaging surface 40A of the imaging table 40. In general, the radiographic image captured by the mammography apparatus 10 is an image of the plane parallel to the imaging surface 40A of the imaging table 40. As described above, in order to easily compare the radiographic image with the ultrasound cross-sectional image that is the plane parallel to the imaging surface 40A of the imaging table 40, the ultrasound cross-sectional image generation unit 74 generates an ultrasound cross-sectional image on the surface parallel to the imaging surface 40A of the imaging table 40. The "substantially parallel" in a case of "plane substantially parallel to the imaging surface 40A of imaging table 40" in the present embodiment refers to that the deflection and inclination of the compression member 34, the inclination of the ultrasound probe 55, and the like are negligible, and that the deflection and inclination of the compression member 34, the inclination of the ultrasound probe 55, and the like are negligible to be regarded as "parallel". In the present embodiment, since a case in which the deflection and inclination of the compression member 34, the inclination of the ultrasound probe 55, and the like are small enough to be negligible will be described, hereinafter, the term "parallel" may be used. In a case in which the deflection and inclination of the compression member 34, the inclination of the ultrasound probe 55, and the like are large to be non-negligible, the "plane substantially parallel to imaging surface 40A of imaging table 40" may be corrected for these.

In a case in which the upper surface 34A of the compression member 34 is parallel to the imaging surface 40A of the imaging table 40 and the inclination of the ultrasound probe 55 during the imaging is ignored, the ultrasound cross-sectional image combined from the plurality of ultrasound images U is a so-called C-mode image and an image of a C surface of the breast. The C-mode image in the ultrasound image is an image of a tomographic plane in a direction perpendicular to the ultrasound beam with respect to a specific constant diagnostic distance. A method in which the ultrasound cross-sectional image generation unit 74 generates the ultrasound cross-sectional image is not limited, and for example, a known technique of reconstructing the plurality of ultrasound images U to reconstruct the cross-sectional image of the surface parallel to the imaging surface 40A of the imaging table 40 can be used.

In the present embodiment, the ultrasound cross-sectional image is described as a two-dimensional cross-sectional image that is the plane parallel to the imaging surface 40A of the imaging table 40 as described above, but the ultrasound cross-sectional image is not limited to a two-dimensional image. The ultrasound cross-sectional image may be, for example, a three-dimensional image having a cross section at any depth and having a thickness in the depth direction.

The ultrasound cross-sectional image generation unit 74 according to the present embodiment generates the ultrasound cross-sectional image corresponding to the plurality of cross sections at any depth. That is, the ultrasound cross-sectional image generation unit 74 according to the present embodiment generates the plurality of ultrasound cross-sectional images. The ultrasound cross-sectional image generation unit 74 outputs the generated ultrasound cross-sectional image to the display control unit 79. In the present embodiment, the ultrasound cross-sectional image generated by the ultrasound cross-sectional image generation unit 74 is an example of an ultrasound image for display according to the present disclosure.

The display control unit 79 superimposes the contour of the breast detected by the breast contour detection unit 73 on the ultrasound cross-sectional image generated by the ultrasound cross-sectional image generation unit 74. The display control unit 79 displays the ultrasound cross-sectional image in a state of being superimposed with the contour of the breast on the display unit 68. In addition, the display control unit 79 according to the present embodiment displays the radiographic image X on the display unit 68.

Next, an operation of the image processing device 18 according to the present embodiment will be described with reference to the drawings, and first, a flow of capturing the radiographic image X and the ultrasound image U using the medical image capturing system 1 will be described. FIG. 6 is a flowchart showing an example of the flow of capturing the radiographic image X and the ultrasound image U using the medical image capturing system 1 according to the present embodiment.

First, the user positions the breast of the examinee as the subject on the imaging surface 40A of the imaging table 40. In a case in which the positioning is completed, the user issues the instruction to compress the breast by the operation unit 26. Therefore, in step S10 of FIG. 6, the control unit 20 of the mammography apparatus 10 starts the compression of the breast by the compression member 34. Specifically, in a case in which the instruction to compress the breast is received, the control unit 20 moves the compression member 34 in the compression direction to compress the breast between the compression member 34 and the imaging surface 40A of the imaging table 40 in the compressed state.

In the subsequent step S12, the mammography apparatus 10 captures the radiographic image X of the breast. Specifically, the user operates an irradiation switch included in the operation unit 26 to irradiate the breast with the radiation R from the radiation source 36R, and the radiographic image X is captured by the radiation detector 30. The radiographic image X captured by the mammography apparatus 10 is output to the console 12, output from the console 12 to the image storage system 19 at a predetermined timing, and stored in the image storage system 19.

In the subsequent step S14, the radiology technician performs the scanning using the ultrasound probe 55 of the ultrasound image capturing apparatus 16 to capture the plurality of ultrasound images U of the breast in the compressed state by the compression member 34. Specifically, after capturing the radiographic image X, the user applies the acoustic matching member (not illustrated) such as echo jelly onto the upper surface 34A of the compression member 34. Further, the user operates the ultrasound probe 55 to scan the upper surface 34A of the compression member 34 covered by the acoustic matching member with ultrasound, and thereby captures the plurality of ultrasound images U.

The ultrasound image captured by the ultrasound image capturing apparatus 16 is displayed on the display unit 58. In addition, the ultrasound image U captured by the ultrasound image capturing apparatus 16 is temporarily stored in the storage unit 52, output from the ultrasound image capturing apparatus 16 to the image storage system 19 at a predetermined timing, and stored in the image storage system 19.

In a case in which the imaging of the ultrasound image U is completed, in the subsequent step S16, the compression of the breast by the compression member 34 is released. Specifically, the user uses the operation unit 26 to give an instruction to release compression. In a case in which the instruction to release the compression of the breast is received, the control unit 20 moves the compression member 34 in the compression release direction to move the compression member 34 in a direction away from the imaging surface 40A of the imaging table 40 to release the compression of the breast by the compression member 34. In this way, in a case in which the process of step S16 ends, the continuous imaging of the radiographic image X and the ultrasound image U ends. In the present embodiment, as in the flow illustrated in FIG. 6, continuously capturing the radiographic image X and the ultrasound image U while the breast W is maintained in a compressed state by the compression member 34 may be referred to as "continuous imaging". An order of the imaging of the radiographic image X (step S12 of FIG. 6) and the imaging of the ultrasound image U (step S14 of FIG. 6) is not limited, but from the viewpoint of shortening the compression time of the breast, it is preferable to perform the imaging of the radiographic image X first as in the present embodiment. In addition, in a case in which the compressed state of the breast is regarded as being unchanged between the imaging of the radiographic image X and the imaging of the ultrasound image U, the compression force with which the breast is compressed by the compression member 34 may be weakened. For example, the compression force with which the breast is compressed by the compression member 34 may be weakened as long as the compression force is regarded as being unchanged in the expansion of the mammary gland of the breast.

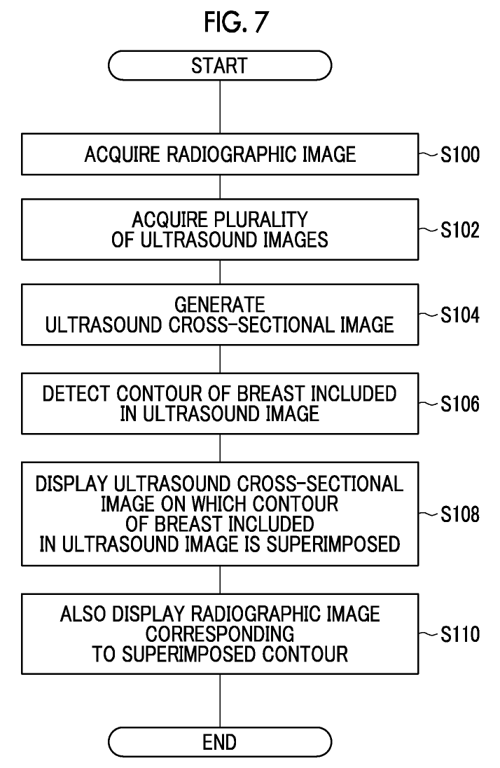

In a case in which the continuous imaging of the radiographic image X and the ultrasound image U ends, the image processing by the image processing device 18 is performed. As an example, the image processing device 18 according to the present embodiment executes the image processing shown in FIG. 7 as an example by the CPU 60A of the control unit 60 executing the image processing program 61 stored in the ROM 60B in a case in which the display condition of the radiographic image X and the ultrasound image U to be displayed, which is input by the user via the operation unit 66, is received. FIG. 7 is a flowchart illustrating an example of a flow of the image processing in the image processing device 18 according to the present embodiment.

First, in step S100, the radiographic image acquisition unit 70 acquires the radiographic image X satisfying the display condition from the image storage system 19 as described above, and outputs the radiographic image X to the breast contour detection unit 73. In the subsequent step S102, the ultrasound image acquisition unit 71 acquires the plurality of ultrasound images U satisfying the display condition from the image storage system 19 as described above, and outputs the plurality of ultrasound images U to the ultrasound cross-sectional image generation unit 74. By the processing of steps S100 and S102, the image processing device 18 acquires the set of the radiographic image X and the ultrasound image U obtained by the continuous imaging.

In the subsequent step S104, the ultrasound cross-sectional image generation unit 74 generates the plurality of ultrasound cross-sectional images corresponding to the cross section at any depth from the plurality of ultrasound images U acquired by the ultrasound image acquisition unit 71 as described above. The ultrasound cross-sectional image generation unit 74 outputs the generated ultrasound cross-sectional image to the display control unit 79.

In the subsequent step S106, the breast contour detection unit 73 detects the contour of the breast included in the radiographic image X as described above, and outputs the detection result and the radiographic image X to the display control unit 79.

In the subsequent step S108, the display control unit 79 displays the ultrasound cross-sectional image on which the contour of the breast included in the radiographic image X is superimposed, on the display unit 68 as described above. Here, the ultrasound cross-sectional image on which the contour of the breast is superimposed, that is, the ultrasound cross-sectional image to be displayed on the display unit 68 is at least any one of the plurality of ultrasound cross-sectional images generated by the ultrasound cross-sectional image generation unit 74. For example, the display control unit 79 receives the designation of the depth of the breast from the person who interprets medical images, selects the ultrasound cross-sectional image corresponding to the received depth of the breast from among the plurality of ultrasound cross-sectional images generated by the ultrasound cross-sectional image generation unit 74, and displays the ultrasound cross-sectional image in a state of being superimposed with the contour of the breast included in the radiographic image X on the display unit 68. In addition, for example, the display control unit 79 displays the ultrasound cross-sectional image having the largest area of the image of the breast among the plurality of ultrasound cross-sectional images generated by the ultrasound cross-sectional image generation unit 74 on the display unit 68 in a state in which the contour of the breast included in the radiographic image X is superimposed.

In the subsequent step S110, the display control unit 79 also displays the radiographic image X corresponding to the contour of the breast superimposed on the ultrasound cross-sectional image on the display unit 68. That is, the radiographic image X acquired in step S100 is also displayed on the display unit 68. In a case in which the processing of step S110 ends, the image processing illustrated in FIG. 7 ends.

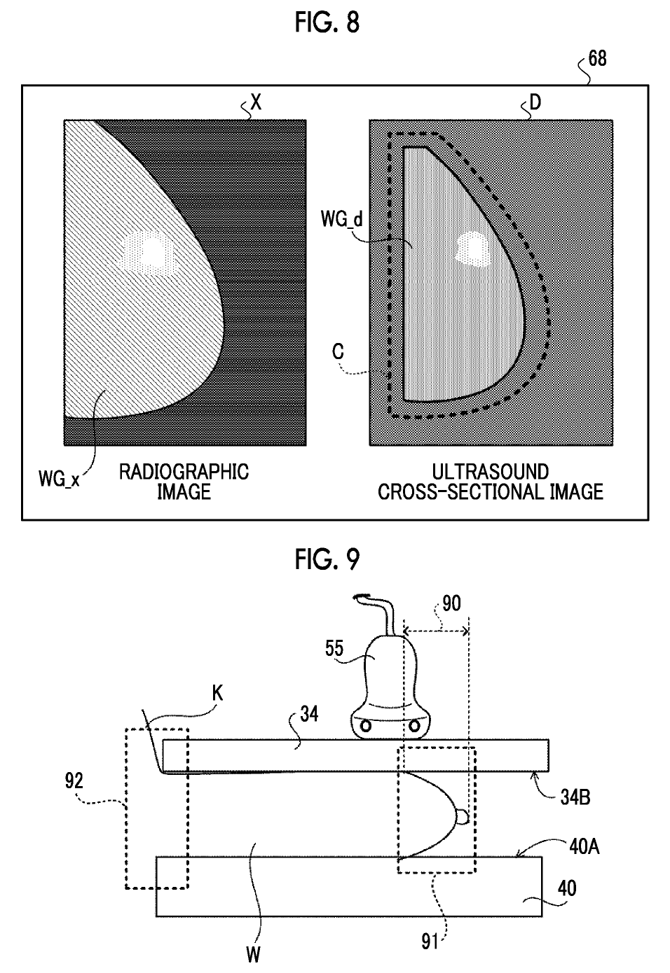

FIG. 8 illustrates an example of a display state of the radiographic image X and the ultrasound cross-sectional image D in a state in which a contour C of the breast included in the radiographic image X is superimposed, the radiographic image X and the ultrasound cross-sectional image D being displayed on the display unit 68 by the display control unit 79. In FIG. 8, the contour C of the breast included in the radiographic image X is larger than a contour of an image WG_d of the breast included in the ultrasound cross-sectional image D. As described above, the reason why the contour C of the breast included in the radiographic image X is larger than the contour of the image WG_d of the breast included in the ultrasound cross-sectional image D will be described with reference to FIGS. 9 to 11.

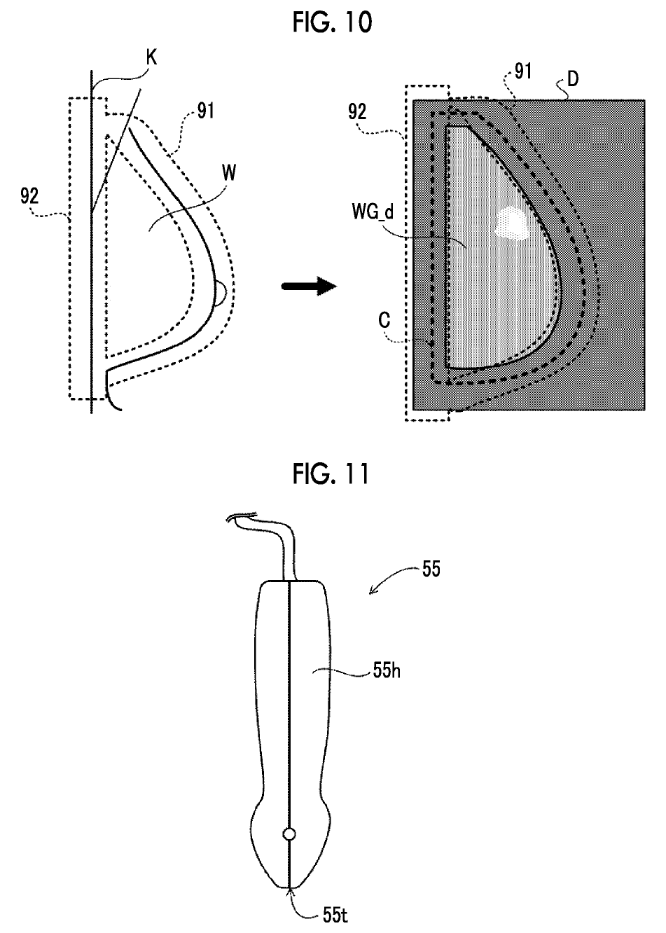

FIG. 9 illustrates a state of the breast W that is in the compressed state between the compression member 34 and the imaging table 40. As illustrated in FIG. 9, in the vicinity of the nipple that is the distal end of the breast, a space may be formed between a lower surface 34B of the compression member 34 and the breast W, and a portion in which the compression member 34 and the breast W are in a non-contact state may be present. In a region 90 in which the compression member 34 and the breast W are in the non-contact state, a state is present in which air is present between the ultrasound probe 55 that scans the compression member 34 and the breast W. Therefore, in the ultrasound image U, the image of the breast is a missing image in the portion corresponding to the region 90. Specifically, as also illustrated in FIG. 10, an image of a missing image region 91 near the skin line of the breast is a missing image in the ultrasound image U.

In addition, as illustrated in FIG. 11, in the ultrasound probe 55, a housing 55h protrudes more than the ultrasound transducer 55t to the periphery. Therefore, the housing 55h may get in the way, and the ultrasound transducer 55t may not reach the vicinity of a chest wall K. In this case, in the ultrasound image U, the image of the missing image region 92 near the chest wall K is the missing image. Specifically, as illustrated in FIGS. 9 and 10, the image of the missing image region 91 on the chest wall K side is the missing image in the ultrasound image U. As described above, since the images of the missing image regions 91 and 92 are the missing images, the image WG_d of the breast of the ultrasound cross-sectional image D is smaller than the image WG_x of the breast of the radiographic image X. Therefore, as illustrated in FIG. 8, the contour C of the breast included in the radiographic image X is larger than the contour of the image WG_d of the breast included in the ultrasound cross-sectional image D.

As described above, in the image processing device 18 according to the present embodiment, as illustrated in FIG. 8, the contour C of the breast included in the radiographic image X is displayed on the display unit 68 in a state in which the contour C of the breast is superimposed on the ultrasound cross-sectional image D. As a result, the person who interprets medical images can interpret medical images while considering the region that is the missing image in the ultrasound cross-sectional image D, that is, a so-called blind area.

In addition, the present embodiment may be modified as follows.

### Modification Example 1

In the present modification example, a case will be described in which the radiographic images X acquired by the radiographic image acquisition unit 70 are the plurality of radiographic images obtained by so-called tomosynthesis imaging.

The mammography apparatus 10 illustrated in FIG. 2 can perform so-called tomosynthesis imaging. The tomosynthesis imaging is an imaging method in which radiation irradiation angles for the subject (breast in the present embodiment) are varied, and the radiographic image X (so called projection image) is captured for each irradiation angle. In a case in which the tomosynthesis imaging is performed, the arm part 42 is rotated, the radiation source 36R of the radiation irradiation unit 36 is moved to each of a plurality of irradiation positions having different irradiation angles (projection angles). For example, the radiation source 36R is moved to the irradiation position at which the irradiation angle varies by a predetermined angle. In other words, the radiation source 36R is moved to a position at which an incidence angle of the radiation R with respect to a detection surface of the radiation detector 30 is different. At each irradiation position, the radiation R is emitted from the radiation source 36R in accordance with the instruction of the console 12, and the radiographic image X is captured by the radiation detector 30. In this way, during the tomosynthesis imaging, a plurality of radiographic images X corresponding to the number of the irradiation positions are obtained. The mammography apparatus that can perform the tomosynthesis imaging may be different from the mammography apparatus 10 illustrated in FIG. 2, and may be, for example, a mammography apparatus provided with the radiation source 36R for each irradiation position and comprising a plurality of radiation sources 36R as the entire apparatus.

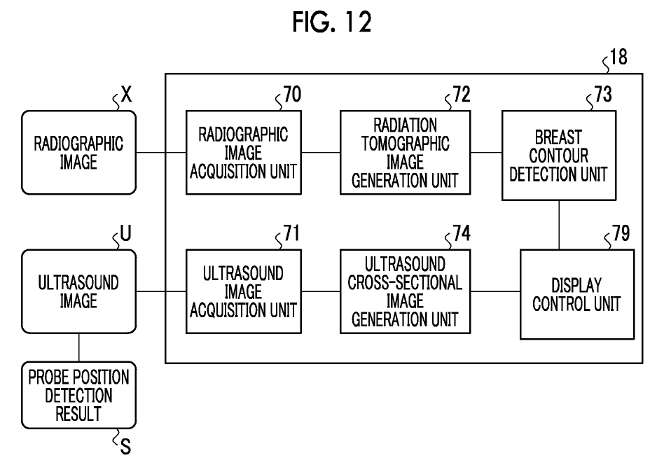

As illustrated in FIG. 12, the image processing device 18 according to the present modification example further comprises a radiation tomographic image generation unit 72 in the image processing device 18 (see FIG. 5) according to the embodiment.

The radiographic image acquisition unit 70 according to the present modification example acquires the plurality of radiographic images X obtained by the tomosynthesis imaging, and outputs the plurality of radiographic images X to the radiation tomographic image generation unit 72.

The radiation tomographic image generation unit 72 generates the plurality of radiation tomographic images corresponding to the plurality of cross sections at different heights from the imaging surface 40A of the imaging table 40 from the plurality of radiographic images X. For example, the number of radiation tomographic images generated by the radiation tomographic image generation unit 72 may be the same as the number of ultrasound cross-sectional images generated by the ultrasound cross-sectional image generation unit 74. As described above, the "depth" in the ultrasound image refers to a depth from the surface (see lower surface 34B in FIG. 9) of the breast in contact with the compression member 34 on the imaging table 40. On the other hand, in the radiation tomographic image, the position of the cross section corresponding to the height direction from the imaging surface 40A is identified with the imaging surface 40A of the imaging table 40 as a reference. In such a case, the radiation tomographic image generation unit 72 converts the height in the radiation tomographic image into the depth in the ultrasound image (ultrasound cross-sectional image). The breast becomes closer to the compression member 34 as the distance from the imaging table 40 becomes longer, so that the depth from the surface of the breast in contact with the compression member 34 becomes shallower as the height from the imaging surface 40A of the imaging table 40 becomes higher.

A method in which the radiation tomographic image generation unit 72 generates the radiation tomographic image from the plurality of radiographic images X is not particularly limited, and a known method can be used. The radiation tomographic image generation unit 72 outputs the plurality of generated radiation tomographic images to the breast contour detection unit 73.

The breast contour detection unit 73 selects the radiation tomographic image at the height corresponding to the ultrasound cross-sectional image for display from among the plurality of radiation tomographic images generated by the radiation tomographic image generation unit 72, detects the contour of the breast included in the selected radiation tomographic image, and outputs the detection result and the selected radiation tomographic image to the display control unit 79.

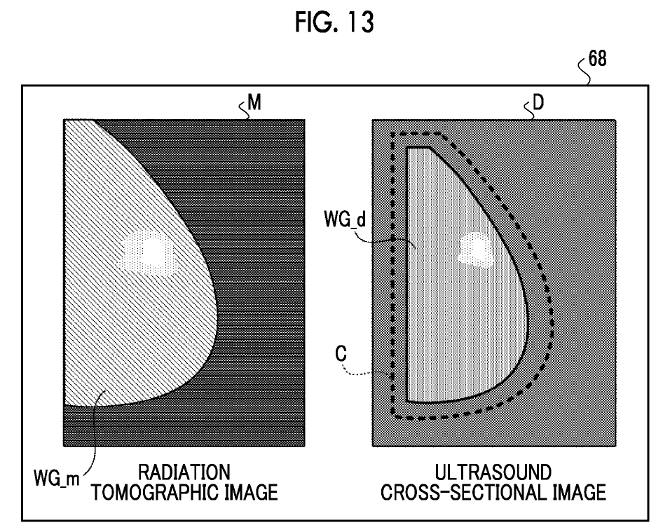

In the present modification example, as illustrated in FIG. 13, the display control unit 79 displays the ultrasound cross-sectional image D on the display unit 68 in a state in which the contour C of the breast included in the radiation tomographic image M at the depth corresponding to the ultrasound cross-sectional image D is superimposed. The contour C of the breast superimposed on the ultrasound cross-sectional image D displayed on the display unit 68 is the same as the outer contour of the image WG_m of the breast included in the radiation tomographic image M. In the example illustrated in FIG. 13, the form is described in which one ultrasound cross-sectional image D is displayed, but a form may be adopted in which a plurality of ultrasound cross-sectional images D at different depths are displayed on the display unit 68. In this case, the display control unit 79 displays each ultrasound cross-sectional image D on the display unit 68 in a state in which the contour C of the breast included in the radiation tomographic image M at the height corresponding to each depth is superimposed.

### Modification Example 2

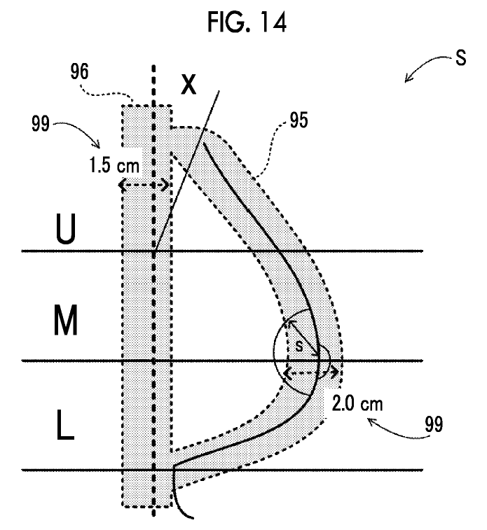

As illustrated in FIG. 14, the display control unit 79 may further display a schematic diagram S in which information 95 and 96 representing a region corresponding to a difference between a region of the breast included in the ultrasound cross-sectional image D to be displayed on the display unit 68 and a region of the breast included in the radiographic image X is added, on the display unit 68. The information 95 is information corresponding to the missing image region 91 in the skin line portion of the breast. Further, the information 96 is information corresponding to the missing image region 92 in the chest wall side portion.

Further, as illustrated in FIG. 14, the display control unit 79 may also display numerical information 99 representing an interval between the contour of the breast included in the ultrasound cross-sectional image D and the contour of the breast included in the radiographic image X.

The present modification example will be specifically described using a case in which the display control unit 79 displays the radiographic image X and the ultrasound cross-sectional image D in a state in which the contour C of the breast included in the radiographic image X is superimposed, as illustrated in FIG. 8, as an example.

First, a method of deriving the information 95 and the numerical information 99 related to the information 95 will be described. The breast contour detection unit 73 according to the present modification example also extracts the contour of the breast included in the ultrasound cross-sectional image D. Then, the breast contour detection unit 73 derives a difference between the contour of the breast included in the radiographic image X and the contour of the breast included in the ultrasound cross-sectional image D as the difference between the region of the breast included in the ultrasound cross-sectional image D and the region of the breast included in the radiographic image X. The breast contour detection unit 73 outputs the difference in the skin line portion of the breast as the information 95 and the numerical information 99 (in FIG. 14, "2.0 cm") to the display control unit 79.

Further, a method of deriving the information 96 and the numerical information 99 related to the information 96 will be described. In this way, the missing image region 92 on the chest wall side occurs in accordance with the housing 55h of the ultrasound probe 55. Therefore, the display control unit 79 acquires information on the ultrasound probe 55 used for capturing the ultrasound image U, specifically, a distance between the outer contour of the housing 55h of the ultrasound probe 55 and the position of the ultrasound transducer 55t. Then, the acquired information on the ultrasound probe 55 is output to the display control unit 79 as the information 95 and the numerical information 99 (in FIG. 14, "1.5 cm").

As illustrated in FIG. 14, the display control unit 79 generates the information 95 the numerical information 99, and the schematic diagram S of the breast in which the information 95 and the numerical information 99 are added, which are input from the breast contour detection unit 73, and causes the display control unit 79 to display the information 95 the numerical information 99, and the schematic diagram S.

As described above, by displaying the schematic diagram S in which the information 95 and 96 and the numerical information 99 are added, the person who interprets medical images can more easily consider the missing image range in the ultrasound image U.

### Modification Example 3

In addition, the ultrasound cross-sectional image displayed on the display unit 68 by the display control unit 79 is not limited to a so-called C-mode image described above. The ultrasound cross-sectional image may be any one of the ultrasound images U obtained by the imaging, which is a first cross-sectional ultrasound image that is a cross-sectional image in a direction intersecting the imaging table 40 on which the breast is placed, and corresponds to a so-called A surface. In addition, the ultrasound cross-sectional image may be a second cross-sectional ultrasound image that is combined from the plurality of ultrasound images U and that is a cross-sectional image in a direction intersecting the imaging table 40 and the ultrasound image U, and corresponds to a so-called B surface. In addition, the type of the ultrasound cross-sectional image to be displayed may be any one of these three types or may be a plurality of types.

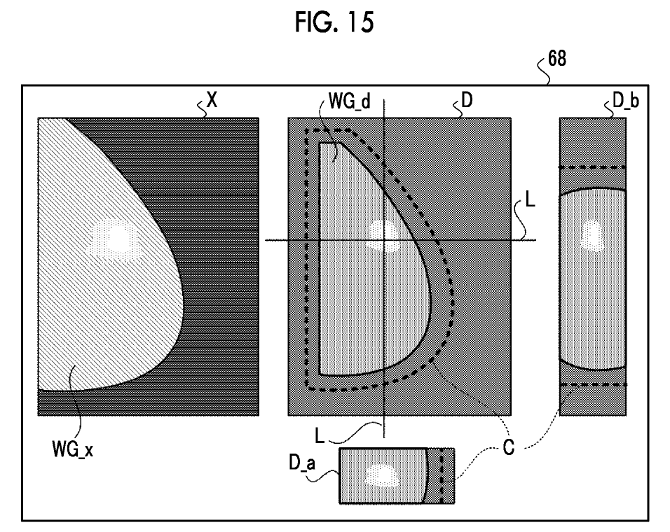

For example, FIG. 15 illustrates a case in which the ultrasound cross-sectional image D_b of the B surface and the ultrasound cross-sectional image D_a of the A surface of the ultrasound cross-sectional image D are displayed in a form in which the radiographic image X and the ultrasound cross-sectional image D in a state in which the contour C of the breast included in the radiographic image X is superimposed are displayed on the display unit 68 as illustrated in FIG. 8. In the example illustrated in FIG. 15, the position of the ultrasound cross-sectional images D_b and D_a to be displayed can be designated by moving the guideline L provided on the ultrasound cross-sectional image D by the person who interprets medical images.

As described above, even in the ultrasound cross-sectional images D_b and D_a, the person who interprets medical images can more easily consider the missing image range in the ultrasound image U by superimposing the contour C of the breast.

As described above, in the image processing device 18 according to the embodiment and each modification example, the radiographic image acquisition unit 70 acquires the radiographic image X captured in the compressed state in which the breast is compressed by the compression member 34. The ultrasound image acquisition unit 71 acquires the plurality of ultrasound images U of the breast in the compressed state that is considered to be the same as the compressed state of the radiographic image X.

The display control unit 79 superimposes the contour of the breast included in the ultrasound cross-sectional image that is combined from the plurality of ultrasound images and that is parallel to the imaging table 40 on the radiographic image X. Further, the display control unit 79 displays the radiographic image X in a state in which the contour of the breast is superimposed.

Therefore, with the image processing device 18 according to the embodiment and each modification example, the person who interprets medical images can intuitively understand the correspondence relationship between the radiographic image and the ultrasound cross-sectional image.

In a case in which the radiographic image X in which the contour of the breast is detected is displayed on the display unit 68, the display control unit 79 may display the radiographic image X in a state in which the contour of the breast included in the ultrasound cross-sectional image D is superimposed. In this case, the display control unit 79 may superimpose the contour of the breast included in the ultrasound cross-sectional image D having the largest region of the breast among the plurality of ultrasound cross-sectional images D on the radiographic image X. In addition, as in the modification example 1, in a case in which the display control unit 79 displays the plurality of radiation tomographic images M in a state in which the contour of the breast included in the ultrasound cross-sectional image D is superimposed, the display control unit 79 may display the plurality of radiation tomographic images M in a state in which the contour of the breast included in the ultrasound cross-sectional image D at the depth corresponding to the height of each radiation tomographic image is superimposed.

In the above embodiment and each modification example, for example, as a hardware structure of a processing unit that executes various types of processing, such as the radiographic image acquisition unit 70, the ultrasound image acquisition unit 71, the radiation tomographic image generation unit 72, the breast contour detection unit 73, the ultrasound cross-sectional image generation unit 74, and the display control unit 79, various processors illustrated below can be used. As described above, the various processors include a programmable logic device (PLD) as a processor whose circuit configuration can be changed after manufacture, such as a field programmable gate array (FPGA), and a dedicated electrical circuit as a processor having a dedicated circuit configuration for executing specific processing such as an application specific integrated circuit (ASIC), in addition to the CPU as a general-purpose processor that functions as various processing units by executing software (program).

One processing unit may be configured by one of these processors, or a combination of two or more processors of the same type or different types (for example, a combination of a plurality of FPGAs, or a combination of a CPU and an FPGA). Also, a plurality of processing units may be configured by one processor.

As an example in which the plurality of processing units are configured by one processor, first, as typified by a computer such as a client or a server, one processor is configured by a combination of one or more CPUs and software, and this processor functions as the plurality of processing units. Second, as typified by a system on a chip (SoC), a processor that realizes the functions of the entire system including the plurality of processing units by using one integrated circuit (IC) chip is used. As described above, as the hardware structure, the various processing units are configured by one or more of the various processors.

Moreover, as the hardware structures of these various processors, more specifically, it is possible to use an electric circuit (circuitry) in which circuit elements, such as semiconductor elements, are combined.

In the above embodiment, the image processing program 61 is described as being stored (installed) in advance in the control unit 60 of the image processing device 18; however, the present disclosure is not limited to this. The image processing program 61 may be provided in a form recorded in recording media such as a compact disc read only memory (CD-ROM), a digital versatile disc read only memory (DVD-ROM), and a universal serial bus (USB) memory. In addition, the image processing program 61 may be downloaded from an external device via a network.

Furthermore, the present disclosure can also be applied to a program and a program product. Specifically, the image processing program 61 in above embodiment may be provided as a program product. The term "program product" encompasses products in any form for providing the program. For example, the program product includes a program provided through a network such as the Internet, and non-transitory computer-readable recording media such as a CD-ROM and a DVD that store the program.

In addition, the configurations and operations of the medical image capturing system 1, the radiographic image capturing system 2, the mammography apparatus 10, the ultrasound image capturing apparatus 16, the image processing device 18, and the like described in the above embodiment and each modification example are examples, and it goes without saying that these can be changed in accordance with the situation within the scope of the present disclosure. Further, it is obvious that the above embodiments may be combined as appropriate.

In regard to the above embodiment, the following supplementary notes will be further disclosed.

### Supplementary Note 1

An image processing device comprising: a processor configured to: acquire a radiographic image captured while a breast is in a compressed state by a compression member; acquire a plurality of ultrasound images captured while the breast is in a compressed state regarded as being the same as the compressed state of the radiographic image by the compression member; and superimpose a contour of the breast included in a radiographic image for superimposition corresponding to the radiographic image on an ultrasound image for display corresponding to the plurality of ultrasound images.

### Supplementary Note 2

The image processing device according to Supplementary Note 1, in which the processor is configured to receive designation of a depth of the breast, and the ultrasound image for display is a cross-sectional image that is combined from the plurality of ultrasound images, that corresponds to the received depth of the breast, and that is substantially parallel to an imaging table.

### Supplementary Note 3

The image processing device according to Supplementary Note 1 or 2, in which the ultrasound image for display is at least one of a first cross-sectional ultrasound image that is any one of the plurality of ultrasound images and that is a cross-sectional image in a direction intersecting an imaging table on which the breast is placed, a second cross-sectional ultrasound image that is combined from the plurality of ultrasound images and that is a cross-sectional image in a direction intersecting the imaging table and the first cross-sectional ultrasound image, or a third cross-sectional ultrasound image that is combined from the plurality of ultrasound images and that is a cross-sectional image substantially parallel to the imaging table.

### Supplementary Note 4

The image processing device according to any one of Supplementary Notes 1 to 3, in which the radiographic image is a projection image obtained by tomosynthesis imaging, and the radiographic image for superimposition is, among a plurality of radiation tomographic images obtained by reconstructing the projection image, the radiation tomographic image at a height corresponding to the ultrasound image for display.

### Supplementary Note 5

The image processing device according to any one of Supplementary Notes 1 to 4, in which the processor is configured to also display the radiographic image for superimposition including the contour of the breast.

### Supplementary Note 6

The image processing device according to Supplementary Note 5, in which the processor is configured to display the radiographic image for superimposition in a state of being superimposed with the contour of the breast included in the ultrasound image for display.

### Supplementary Note 7

The image processing device according to any one of Supplementary Notes 1 to 6, in which the processor is configured to further display a schematic diagram in which information representing a region corresponding to a difference between a region of the breast included in the ultrasound image for display and a region of the breast included in the radiographic image for superimposition is added.

### Supplementary Note 8

The image processing device according to Supplementary Note 7, in which the information includes a numerical value representing an interval between the contour of the breast included in the ultrasound image for display and the contour of the breast included in the radiographic image for superimposition.

### Supplementary Note 9

A medical image capturing system comprising: the image processing device according to any one of Supplementary Notes 1 to 8; a radiographic image capturing apparatus; and an ultrasound image capturing apparatus.

### Supplementary Note 10

An image processing method executed by a processor provided in an image processing device, the image processing method comprising: acquiring a radiographic image captured while a breast is in a compressed state by a compression member; acquiring a plurality of ultrasound images captured while the breast is in a compressed state regarded as being the same as the compressed state of the radiographic image by the compression member; and superimposing a contour of the breast included in a radiographic image for superimposition corresponding to the radiographic image on an ultrasound image for display corresponding to the plurality of ultrasound images.

### Supplementary Note 11

An image processing program causing a processor provided in an image processing device to execute a process comprising: acquiring a radiographic image captured while a breast is in a compressed state by a compression member; acquiring a plurality of ultrasound images captured while the breast is in a compressed state regarded as being the same as the compressed state of the radiographic image by the compression member; and superimposing a contour of the breast included in a radiographic image for superimposition corresponding to the radiographic image on an ultrasound image for display corresponding to the plurality of ultrasound images.

The disclosure of Japanese Patent Application No. 2023-170679, filed on Sep. 29, 2023, is incorporated in this specification by reference in its entirety. All of the documents, the patent applications, and the technical standards described in this specification are incorporated into this specification by reference to the same extent as in a case in which each of the documents, the patent applications, and the technical standards are specifically and individually stated to be described by reference.

What is claimed is:

1. An image processing device comprising a processor configured to:
acquire a radiographic image captured while a breast is in a compressed state by a compression member;
acquire a plurality of ultrasound images captured while the breast is in a compressed state regarded as being the same as the compressed state of the radiographic image by the compression member; and
superimpose a contour of the breast included in a radiographic image for superimposition corresponding to the radiographic image on an ultrasound image for display corresponding to the plurality of ultrasound images.

2. The image processing device according to claim 1, wherein:
the processor is configured to receive designation of a depth of the breast, and
the ultrasound image for display is a cross-sectional image that is combined from the plurality of ultrasound images, that corresponds to the received depth of the breast, and that is substantially parallel to an imaging table.

3. The image processing device according to claim 1, wherein the ultrasound image for display is at least one of:
a first cross-sectional ultrasound image that is any one of the plurality of ultrasound images and that is a cross-sectional image in a direction intersecting an imaging table on which the breast is placed,
a second cross-sectional ultrasound image that is combined from the plurality of ultrasound images and that is a cross-sectional image in a direction intersecting the imaging table and the first cross-sectional ultrasound image, or
a third cross-sectional ultrasound image that is combined from the plurality of ultrasound images and that is a cross-sectional image substantially parallel to the imaging table.

4. The image processing device according to claim 1, wherein:
the radiographic image is a projection image obtained by tomosynthesis imaging, and
the radiographic image for superimposition is, among a plurality of radiation tomographic images obtained by reconstructing the projection image, the radiation tomographic image at a height corresponding to the ultrasound image for display.

5. The image processing device according to claim 1, wherein the processor is configured to also display the radiographic image for superimposition including the contour of the breast.

6. The image processing device according to claim 5, wherein the processor is configured to display the radiographic image for superimposition in a state of being superimposed with the contour of the breast included in the ultrasound image for display.

7. The image processing device according to claim 1, wherein the processor is configured to further display a schematic diagram in which information representing a region corresponding to a difference between a region of the breast included in the ultrasound image for display and a region of the breast included in the radiographic image for superimposition is added.

8. The image processing device according to claim 7, wherein the information includes a numerical value representing an interval between the contour of the breast included in the ultrasound image for display and the contour of the breast included in the radiographic image for superimposition.

9. A medical image capturing system comprising:
the image processing device according to claim 1;
a radiographic image capturing apparatus; and
an ultrasound image capturing apparatus.

10. An image processing method executed by a processor provided in an image processing device, the image processing method comprising:
acquiring a radiographic image captured while a breast is in a compressed state by a compression member;
acquiring a plurality of ultrasound images captured while the breast is in a compressed state regarded as being the same as the compressed state of the radiographic image by the compression member; and
superimposing a contour of the breast included in a radiographic image for superimposition corresponding to the radiographic image on an ultrasound image for display corresponding to the plurality of ultrasound images.

11. A non-transitory computer-readable storage medium storing an image processing program causing a processor provided in an image processing device to execute a process comprising:
acquiring a radiographic image captured while a breast is in a compressed state by a compression member;
acquiring a plurality of ultrasound images captured while the breast is in a compressed state regarded as being the same as the compressed state of the radiographic image by the compression member; and
superimposing a contour of the breast included in a radiographic image for superimposition corresponding to the radiographic image on an ultrasound image for display corresponding to the plurality of ultrasound images.
